(19) United States
(12) Patent Application Publication
(10) Pub. No.: US 2010/0185237 A1
(43) Pub. Date: Jul. 22, 2010

(54) DEVICE FOR ATTACHING SUTURE THREADS

(76) Inventor: Yves-Alain RATRON, Grenoble (FR)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/727,477

(22) Filed: Mar. 19, 2010

Related U.S. Application Data
(63) Continuation of application No. PCT/FR2008/051683, filed on Sep. 19, 2008.

(30) Foreign Application Priority Data
Sep. 21, 2007 (FR) 0757763

(51) Int. Cl. A61B 17/04

(52) U.S. Cl. 606/232

(57) ABSTRACT

An embodiment of a device for fixation of a suture thread includes a fixation element with a base and a hole, the fixation element including a first surface for placement against the structure, and a second surface opposite the first surface. Such an embodiment may further include an anchoring element for anchoring within the structure, which includes a head and a stem, the stem configured for placement through the hole with the head facing the second surface, wherein the fixation element includes a locking element comprising an aperture configured to receive the suture thread, the aperture having an unlocked configuration permitting free passage of the suture and a locked configuration inhibiting suture movement, wherein the locking element is located at least partially between the head and the second surface, such that the head of the anchoring element compresses the locking element from the unlocked configuration into the locked configuration.

### CROSS-REFERENCE TO RELATED APPLICATIONS

This application is a continuation of Patent Cooperation Treaty Application Number PCT/FR2008/051683, filed on Sep. 19, 2008, which claims foreign priority to French Patent Application Number 0757763, filed on Sep. 21, 2007.

### TECHNICAL FIELD

This invention concerns a device for attaching suture threads to a structure, on a bone or a prosthetic element in particular.

### BACKGROUND

In the medical field, the use of a suture anchor to fasten soft tissue is known, such as a tendon or a ligament, to a bone. In a traditional fashion, a suture anchor includes a support element on the bone, associated to a screw or an anchoring stud in the bone. Prior to the total deflection of the screw or the stud into the bone, a suture thread is introduced and wrapped between the head of the screw or the stud and the support element. The suture thread is then locked into place in the suture anchor by fully screwing the stud or the screw into the bone, so that it compresses the suture thread between the head of the screw or stud and the support element. The set up of this suture anchor makes it difficult to wind the suture thread on the screw or the stud, of which only a minimal portion projects from the surface of the bone. Moreover, it is not possible to preassemble this type of suture anchor, and for this reason it cannot be implanted arthroscopically.

Moreover, it is known from U.S. Pat. No. 5,669,917 to join two tissues using suture threads maintained at a desired tension with the help of a clamping ring. Initially, the clamping ring is cylindrical and suitable for bending using the action of an appropriate instrument so as to immobilize the suture threads in its interior volume. This type of clamping ring for suture threads cannot be used to fasten soft tissue onto a bone. In effect, fastening suture threads to bone requires the presence of an anchoring structure in the bone. Moreover, the clamping ring around the suture threads requires setting up a complex instrument to bend the ring arthroscopically.

### SUMMARY

In particular, the invention seeks to remedy these disadvantages by proposing a device for fastening suture threads onto a structure, such as a tissue or a prosthetic element, the implementation of which is easy and feasible during an arthroscopic intervention, without requiring a specific instrument.

For this purpose, the invention has for its object a fastening device for suture threads on a structure, such as a tissue or a prosthetic element, including:
a fixation element comprising a base formed with a central bore, the bore extending between a first and a second surface opposite the base, the first surface being adapted to rest against the structure, and
an anchoring element in the structure, including a head and a stem, the stem being adapted to be received in the bore of the base with the head facing the second surface,
characterized in that the fastening element includes at least one tubular component defining a housing for the passage of at least one suture thread, the tubular component extending at least partially in relation with the second surface of the base with its central axis substantially perpendicular to the central axis of the bore, the head of the anchoring element being able to at least locally compress the tubular component, with an induced reduction of the housing area from an area permitting free passage of a suture thread to an area that locks the suture thread, when anchoring the anchoring element in the structure with the first surface of the support base against the structure and the stem of the anchoring element received in the bore.

In the sense of the invention, the tubular component shall be understood as a component having the form of a tube, with a transverse section which may be circular or not, the peripheral wall of the tube being continuous or with a local longitudinal opening with respect to the tube, of which the length is shorter than the perimeter of the tube, and in particular shorter than about 20% of the tube perimeter.

According to other advantageous characteristics of the invention, taken separately or all the technically possible combinations:
each tubular component has a peripheral wall which is able to be bent by the head of the anchoring element during the anchoring of the anchoring element in the structure with the first surface of the base supported against the structure and the stem of the anchoring element received in the bore;
the fastening element includes two tubular components diametrically opposed on both sides of the bore;
each tubular component is derived from the material of the base; each tubular component is a strip folded so as to define the housing;
each tubular component is an extra thickness of the base pierced so as to define the housing;
each tubular component is a tube added onto the second face of the base;
the first surface of the base comprises means to block rotation of the fastening element with respect to the structure;
the anchoring element is a screw.

Another object of the invention is a process of fastening suture threads on a structure, such as a tissue or a prosthetic element, through the use of a fastening device such as that described above, this procedure includes the following stages in which:
a portion of at least one suture thread is introduced into the housing of each tubular component on the fastening element;
the fastening device is positioned with respect to the structure by anchoring the anchoring element in the structure with the first surface of the support base against the structure and the stem of the anchoring element received into the bore in the base;
each suture thread is tightened;
the anchoring element is anchored into the structure until the anchoring element reaches a position of maximum anchorage, in which the head compresses each tubular component so that the section of the housing defined by the tubular component is the section that locks in a suture thread.

### BRIEF DESCRIPTION OF THE DRAWINGS

The characteristics and advantages of the invention will appear in the following description of three embodiments of a suture thread fastening device in accordance with the invention, which are only given as an example and are made in reference to the drawings attached in which:

FIG. 1 is a perspective view of a fastening device in accordance with a first embodiment of the invention, during a first stage for fastening suture threads to a bone, the suture threads being represented by mixed broken lines for better visibility;

FIG. 2 is a cross sectional view according to plane II of FIG. 1;

FIG. 3 is an analogous cross sectional view of FIG. 2, during the second stage of fastening the suture threads on a bone using the fastening device of FIG. 1;

FIG. 4 is a perspective view analogous to FIG. 1 for a suture thread fastening device in accordance with a second embodiment of the invention; and

FIG. 5 is a perspective view analogous to FIG. 1 for a suture thread fastening device in accordance with a third embodiment of the invention.

### DETAILED DESCRIPTION

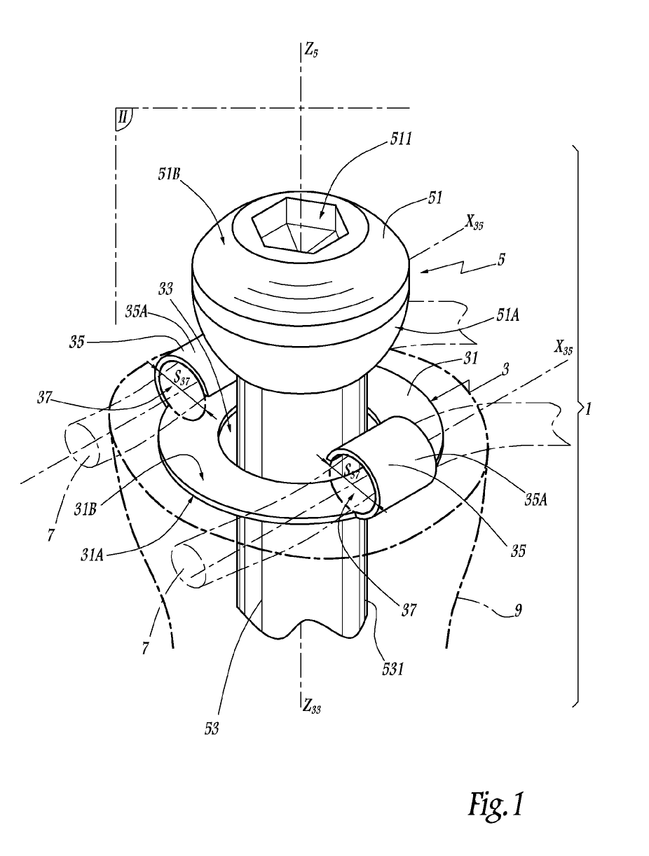

The suture thread fastening device (1) shown in FIGS. 1 to 3 is designed to be mounted on a bone (9), for example, fastening a soft tissue such as a tendon or a ligament on this bone.

Device 1 includes a fastening washer (3) and an anchoring screw (5) in the bone (9), made of a biocompatible material. The washer (3) and screw (5) may also be made of a bioabsorbible material. The washer (3) includes a base (31) having a substantially circulare cross section, the first surface (31A) of which is designed to be supported against the bone (9). Base (31) is pierced with a central bore (33) having axis Z₃₃. The washer (3) also includes two tubes (35) diametrically opposed on both sides of the bore (33), each tube (35) projecting with respect to the second surface (31B) of the base (31) opposed to the surface (31A) and defining a housing (37) for the passage of a suture thread (7). It may be observed that the central axis X₃₅ of each tube (35), is substantially perpendicular to the central axis Z₃₃ of the bore (33). Each tube (35) is formed by a strip, derived from the base (31) material and is folded over itself on the side of the surface (31B) so as to define the housing (37).

The screw (5) of the device (1), whose longitudinal axis Z₅ may be observed, includes a head (51) and a stem (53). The screw (5) is designed to be mounted on the washer (3) by introducing the stem (53) into the bore (33) with the head (51) facing the surface (31 B), the axes Z₃₃ and Z₅ being substantially merged. As is shown in the figures, the screw (5) is a self-tapping screw, the stem (53) being provided with an external thread (531). Moreover, the head (51) is provided with a hollow polygonal housing (511) to receive an operating key (not shown) at the level of its surface (51B) opposite the stem (53), making it possible to turn the screw (5) around the axis Z₅.

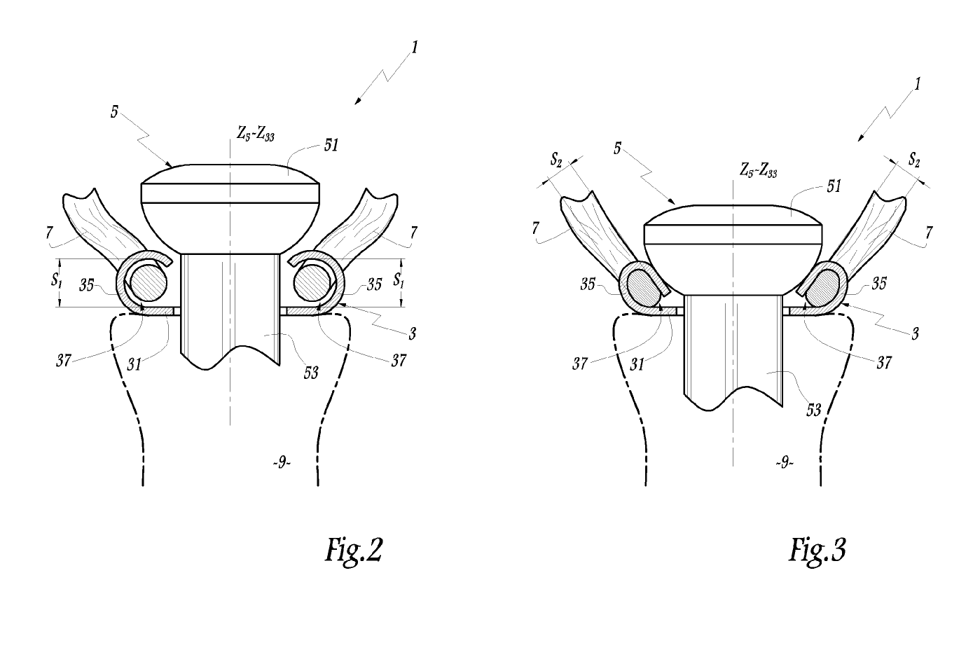

Prior to mounting the device (1) on the bone (9), the S₃₇ section of the housing (37) of each tube (35) has a value represented in FIG. 2 by dimension S₁, the housing (37) allowing free passage of the suture thread (7) into the tube (35). The peripheral wall (35A) of each tube (35), having a small thickness, is able to be bent by the head (51) of the screw (5), during anchoring accomplished by screwing the screw into the bone (9) with the surface (31A) of the base (31) supported against the structure (9) and the stem (53) received into the bore (33). More specifically, the surface (51A) of the head (51) directed towards the stem (53) is able to simultaneously compress the two tubes (35) during anchoring by screwing in the screw (5) into the bone (9), which involves a reduction of the section (S₃₇) of the housing (37) of each of two tubes (35). In the maximum docking position of the screw (5) in the bone (9), the washer (3) is immobilized between the bone (9) and the surface (51A) of the head (51) of the screw (5). In this maximum anchoring position, section S₃₇ of the housing (37) of each tube (35) has been assigned a value, shown in FIG. 3 by the dimension S₂, which is lower than the value shown by dimension S₁, the suture thread (7) passing through the housing (37) formed of each tube (35) which is then locked with respect to the washer (3), which is itself immobilized at the surface of the bone (9). The device (1) according to the invention thus also allows immobilization of suture threads (7) received in the housings (37) of the tubes (35), simultaneous with the anchoring of the screw (5) into the bone (9).

According to the method of intervention for implantation of the device (1), the device (1) may be placed onto the bone (9) with or without preassembling the washer (3) and the anchoring screw (5). A process for fastening suture threads (7) on the bone (9) using the device (1) includes the steps in which:

First of all, a portion of suture thread (7) is introduced into the housing (37) of each of the two tubes (35) of washer (3).

Then the washer (3) is positioned, fitted with suture threads (7), with respect to the bone (9). For this purpose, the washer (3) is placed to rest against the surface of the bone (9), then the stem (53) of the screw (5) is introduced into the bore (33) and the screw (5) is anchored by screwing it into the bone (9) using an operating key (not shown) working from the housing (511) of the head (51). For example, screwing in the screw (5) may be carried out until the surface (51A) of the head (51) comes into contact with the tubes (35) of the washer (3), without compressing the tubes (35).

When the device (1) is thus positioned with respect to the bone (9), the suture threads (7) received in the housings (37) are tightened, to a tension adapted to their function.

The screw (5) is then anchored more tightly into the bone (9), using the screw, so as to immobilize the device (1) with respect to the bone (9). The screw (5) is screwed in until it reaches a position of maximum anchoring, in which the head (51) compresses each tube (35), such that the section (S₃₇) of the housing (37) of each tube (35) reaches the value shown by dimension S₂, the suture threads (7) being then locked in the tubes (35).

From this fastening process, described above, the device (1) in accordance with the first embodiment, allows easy implantation of the device on the bone (9), the suture threads (7) being locked in place with respect to the washer (3) at the same time as the screw (5) is anchored into the bone (9) for the immobilization of the washer (3) on the bone.

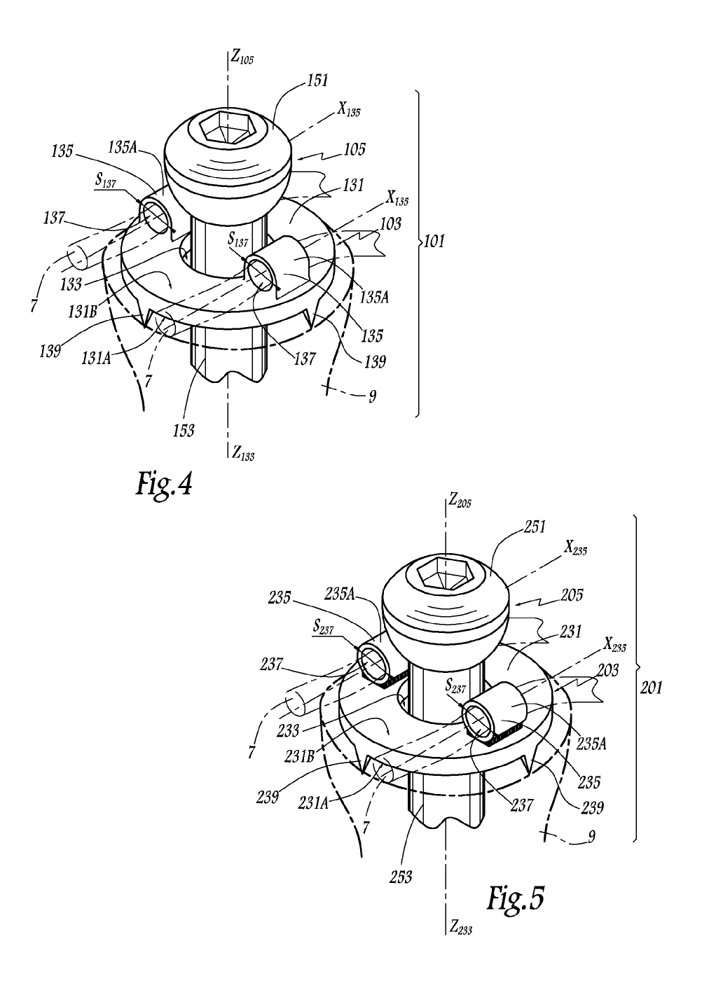

In the second embodiment shown in FIG. 4, the elements similar to those in the first embodiment use an identical reference system increased by 100. As shown in FIG. 4, the suture thread (7) fastening device (101), according to this second embodiment, is mounted on a bone (9). The device (101) includes a washer (103), the base (131) of which is intended to rest against the surface of the bone (9), at the level of one of its surfaces (131A). The device (101) also includes an anchoring screw (105) in the bone (9), the longitudinal axis Z₁₀₅ of which is noted. A stem (153) of the screw (105) is adapted to be introduced into a bore (133) formed in the base (131) of washer (103), the head (151) of the screw (105) facing the surface (131B) of the base (131) opposite to the surface (131A).

The washer (103) includes two tubes (135) intended for the passage of the suture threads (7) and diametrically opposed on one side and another of the bore (133), the central axis X₁₃₅ of each tube (135) being substantially perpendicular to the central axis Z₁₃₃ of the bore (133). Each tube (135) is an extra thickness of the base (131) projecting with respect to the surface (131B), pierced with a housing (137) for the passage of a suture thread (7). The peripheral wall (135A) of each tube (135) has a relatively low thickness, such that the wall is capable of being bent by the head (151) of the screw (105) during anchoring by screwing it into the bone (9) with the surface (131A) resting against the bone (9) and the screw (153) received in the bore (133).

In the second embodiment, the surface (131A) of the base (131) is provided with barbs (139) intended to be pressed into the upper layer of the bone (9). Thus, when the washer (103) is pressed into the bone (9), the barbs (139) are able to block the rotation with respect to the bone (9), so as to permit anchoring of the screw (105) by screwing it into the bone (9) without rotational movement of the washer (103), which is advantageous for obtaining an efficient compression of the tubes (135) near the position of maximum anchorage of the screw (105). Thus the device (105) allows for effective locking of the suture thread (7) in the tubes (135) due to the compression of at least part of these tubes by the head (151), with an induced reduction of the section S₁₃₇ of the housing (137) of each tube (135), from a section permitting the free passage of a suture thread (7), to a section locking the suture thread, during anchoring accomplished by screwing the screw (105) into the bone (9), with the washer (103) immobilized from rotating on the bone (9) due to the barbs (139) and the stem (153) received in bore (133).

In the third embodiment shown in FIG. 5, the elements similar to those in the first embodiment use an identical reference system increased by 200.

The device (201) in accordance with this third embodiment is adapted to be mounted on a bone (9) and includes a washer (203) which rests on the bone (9) and an anchoring screw (205) in the bone (9), the longitudinal axis Z₂₀₅ of which is noted. The stem (253) of the screw (205) is intended to be introduced into a bore (233) formed in the base (231), the head (251) of the screw (205) facing the surface (131B) of the base (231) opposite the surface (231A) resting on the bone (9). In this third embodiment, the washer (203) includes two tubes (235) attached by soldering on the surface (231B) of the base (231), the tubes (235) being diametrically opposed on one side and the other of the bore (233), with their central axes X₂₃₅ substantially perpendicular to the central axis Z₂₃₃ of the bore (33) and parallel to each other. As in the other two prior embodiments, each tube (235) defines a housing (237) for passage of a suture thread (7), the anchoring by screwing the screw (205) into the bone (9) allowing at the same time immobilization of the washer (203) between the bone (9) and the head (251) of the screw (205) and an immobilization of the suture threads (7) in the tubes (235), due to the compression of these tubes by the head (251). The peripheral wall (235A) of each tube (235) is made of a relatively flexible material, of the type that will make this wall suitable to be bent by the head (251) when anchoring the screw (205) by screwing it into the bone (9) with the surface (231A) resting against the bone (9) and the stem (253) received by the bore (233), with an induced reduction of the section S₂₃₇ of the housing (237) of each tube (235) from a section permitting free passage of a suture thread (7) to a section that locks the suture thread, when the screw (205) is in a maximum anchorage position in the bone (9). As in the second embodiment, the base (231) of the washer (203) also includes barbs (239) for blocking rotation of the washer (203) with respect to the bone (9).

In the Figures attached hereto, for clearness of the drawing, sections S₃₇, S₁₃₇ et S₂₃₇, are circular and are distinguished by their respective diameters. However, these sections may not be circular.

As arises from the embodiments described above, a suture thread fastening device in accordance with the invention allows an easy implementation of the device on a structure, for example on a bone, the suture threads being received within the tubes of the washer and immobilized in these tubes near a maximum anchoring position of the screw within the structure, due to compression of the tubes by the head of the screw. In particular, the compression of the tubes for locking the suture threads does not require any specific instrument.

Moreover, by preassembling the washer fitted with suture threads and the screw by any appropriate means, a suture thread fastening device according to the invention may be utilized arthroscopically. In fact, the insertion of the suture threads into the tubes of the washer prior to implantation of the device on a structure permits easy implementation of a device in accordance with the invention arthroscopically, by eliminating the step of positioning the suture threads between the head of the screw and the support element, which is a necessary step with state-of-the-art suture anchors and is difficultly achieved with arthroscopic interventions.

The invention is not limited to the examples described and shown. In particular, the anchoring element of a suture thread fastening device according to the invention may be of any appropriate type other than a screw. In the event that the anchoring element is a screw, it may not be a self-tapping screw. When the screw of a fastening device in accordance with the invention is not a self-tapping screw, a perforated housing is formed in the structure so as to receive the stem of the screw prior to installation of the device on the structure.

Moreover, the number of tubes planned on the washer of a suture thread fastening device according to the invention may be different from two. In an advantageous manner, the tubes are diametrically opposed in pairs on one side and the other of the bore in the washer, so that the anchoring element in the structure is not deviated with respect to the central axis of the device in the course of anchoring, and in particular when nearing the maximum anchoring position, during the crushing of the tube. Moreover, each tube of the washer of a fastening device according to the invention may be set up for the passage of several suture threads, the number of these suture threads may be optimized in terms of application.

As shown in the embodiments described, the presence of barbs to block rotation of the washer of the fastening device in accordance with the invention is not mandatory, but is advantageous for favoring the compression of the tubes of the washer when nearing the maximum anchoring position of the anchoring element, in avoiding any involvement of rotation in the washer by contact between the head of the anchoring element and the peripheral wall of the tubes. These barbs may be seen on the washer (3) in the first embodiment.

Finally, a suture thread fastening device in accordance with the invention may also be used to fasten suture threads on a bone covered with a soft tissue, the anchoring element being then designed with a length sufficient to penetrate the bone. Moreover, a suture thread fastening device in accordance with the invention may be used during the positioning of an implant, such as an osteosynthesis plate.

What is claimed is:

1. A device for fixation of a suture to a structure, comprising:
a fixation element comprising a base and a hole formed through the base, the fixation element comprising a first surface configured to be placed against the structure, and a second surface opposite the first surface;
an anchoring element configured for anchoring within the structure, the anchoring element comprising a head and a stem, the stem configured for placement through the hole such that the head faces the second surface;
wherein the fixation element comprises a locking element, wherein the locking element comprises an aperture configured to receive the suture, the aperture having an unlocked configuration permitting free passage of the suture through the aperture and a locked configuration in which movement of the suture through the aperture is inhibited, and
wherein the locking element is located at least partially between the head and the second surface, such that anchoring the anchoring element into the structure through the hole compresses the locking element from the unlocked configuration into the locked configuration.

2. The device of claim 1, wherein the fixation element is a washer.

3. The device of claim 1, wherein the first surface comprises one or more barbs configured to inhibit rotation of the anchoring element with respect to the structure.

4. The device of claim 1, wherein the structure is a bone.

5. The device of claim 1, wherein the locking element is a tube.

6. The device of claim 5, wherein the tube comprises a peripheral wall adapted to be bent by the head of the anchoring element as the anchoring element is anchored into the structure through the hole.

7. The device of claim 1, wherein the locking element is a first locking element, and the aperture is a first aperture, the device further comprising a second locking element, wherein the second locking element comprises a second aperture configured to receive the suture, the second aperture having an unlocked configuration permitting free passage of the suture through the aperture and a locked configuration in which movement of the suture through the aperture is inhibited, and wherein the second locking element is located at least partially between the head and the second surface, such that anchoring the anchoring element into the structure through the hole compresses the second locking element from the unlocked configuration into the locked configuration.

8. The device of claim 7, wherein the first locking element and the second locking element are diametrically opposed to each other on opposite sides of the hole.

9. The device of claim 1, wherein the locking element is integral with the anchoring element.

10. The device of claim 7, wherein the first and second locking elements are integral with the anchoring element.

11. The device of claim 1, wherein the locking element is formed of the same material as the anchoring element.

12. The device of claim 1, wherein the anchoring element is a screw.

13. The device of claim 12, wherein the screw is a self-tapping screw.

14. The device of claim 1, wherein the locking element is a strip folded so as to define the aperture.

15. The device of claim 1, wherein the locking element comprises an extra thickness of the fixation element pierced so as to define the aperture.

16. The device of claim 1, wherein the locking element is connected to the second surface.

17. A method for fastening a suture onto a structure, comprising:
placing a first surface of a fixation element against the structure, the fixation element comprising a hole formed therethrough and a second surface opposite the first surface;
inserting an anchoring element through the hole, the anchoring element comprising a head and a stem;
inserting the stem into the structure; placing the suture through an aperture in a locking element of the fixation element; and
compressing the aperture of the locking element by anchoring the anchoring element into the structure through the hole, to substantially inhibit movement of the suture within the locking element.

18. The method of claim 17, wherein the anchoring element is a screw, and wherein compressing the aperture further comprises driving the screw through the hole into the structure until a head of the screw compresses the locking element.

19. The method of claim 18, wherein compressing the locking element comprises compressing the locking element between the head and the second surface.
